(19) United States
(12) Patent Application Publication
(10) Pub. No.: US 2014/0187992 A1
(43) Pub. Date: Jul. 3, 2014

(54) SYSTEM AND METHOD FOR NON-INVASIVELY DETERMINING CARDIAC OUTPUT

(71) Applicant: COVIDIEN LP, Boulder, CO (US)

(72) Inventor: Tom Wilmering, Westminster, CO (US)

(73) Assignee: Covidien LP, Boulder, CO (US)

(21) Appl. No.: 13/733,436

(22) Filed: Jan. 3, 2013

(51) Int. Cl. A61B 5/029

(52) U.S. Cl. CPC A61B 5/029
USPC 600/526

(57) ABSTRACT

A system for non-invasively determining cardiac output of a patient may include a physiological signal detection unit and a cardiac output determination module. The physiological signal detection unit may be configured to detect first and second physiological signals with respect to first and second locations of vasculature of the patient. The cardiac output determination module may be configured to receive the first and second physiological signals and calculate the cardiac output of the patient based, at least in part, on a phase difference between the first and second physiological signals.

### FIELD

Embodiments of the present disclosure generally relate to physiological signal processing and, more particularly, to a system and method for non-invasively determining the cardiac output of a patient through an analysis of one or more physiological signals.

### BACKGROUND

Determination of cardiac output represents an accurate assessment of overall cardiovascular health of an individual. Cardiac output, or blood volumetric flow rate, relates to the volume of blood pumped by a heart over time, such as per minute. In general, cardiac output is a function of heart rate and stroke volume. The heart rate is the number of heart beats per minute, while the stroke volume is the volume of blood pumped out of the heart with each beat (that is, during each contraction). An increase in either heart rate or stroke volume increases cardiac output. Overall, a determination of cardiac output allows an individual to monitor central circulation. Additionally, such a determination provides improved insights into normal physiology, pathophysiology, and treatments for disease.

Cardiac output may be measured in various ways. For example, cardiac output may be measured invasively, such as through implanting a device, such as a cardiac catheter, into vasculature of a patient. However, invasive techniques typically require surgery in order to implant the device within the vasculature. The surgical operation may be painful to the patient, and also labor-intensive and risky. As such, non-invasive methods of determining cardiac output have been developed. Typically, however, noninvasive methods may not always be quickly and easily performed, or entirely accurate. As an example, blood pressure signals may be used to determine a cardiac output of a patient. In general, a parameter derived solely from the blood pressure waveform may be used with respect to a predictive model in order to yield information related to cardiac output. However, the predictive model may not account for variable physiological parameters. As such, determining cardiac output by analyzing a blood pressure signal or waveform may lead to erroneous predictions regarding cardiac output, which may, in turn, lead to false diagnoses, for example.

### SUMMARY

Certain embodiments of the present disclosure provide a system for non-invasively determining cardiac output of a patient that may include a physiological signal detection unit and a cardiac output determination module. The physiological signal detection unit is configured to detect first and second physiological signals with respect to first and second locations of vasculature of the patient. The cardiac output determination module is configured to receive the first and second physiological signals and calculate the cardiac output of the patient based, at least in part, on a phase difference between the first and second physiological signals.

The physiological signal detection unit may include a light emitter and first and second photodetectors. The first and second photodetectors are configured to detect the first and second physiological signals, respectively. The light emitter and the first and second photodetectors may be configured to align with the vasculature of the patient. The first and second photodetectors may be equidistant from the light emitter.

Each of the first and second physiological signals may include a photoplethysmography (PPG) signal. In an embodiment, the physiological signal detection unit includes a pulse oximetry sensor.

The physiological signal detection unit may include a housing defining an internal chamber configured to receive a portion of a finger. In another embodiment, the physiological signal detection unit may include a strap configured to be positioned on an anatomical portion of the patient. In another embodiment, the physiological signal detection unit may include one or more of a headband or a headset. In still another embodiment, the physiological signal detection unit may include a sleeve configured to be positioned around a portion of an arm or a leg.

Certain embodiments of the present disclosure provide a method of non-invasively determining cardiac output of a patient. The method may include positioning a physiological signal detection unit with respect to an anatomical portion of the patient, emitting light from a light emitter of the physiological signal detection unit into vasculature proximate to the anatomical portion, detecting first and second physiological signals with first and second photodetectors, respectively, positioned in relation to first and second locations of the vasculature, receiving the first and second physiological signals at a cardiac output determination module, using the cardiac output determination module to determine a phase difference between the first and second physiological signals, and calculating, with the cardiac output determination module, the cardiac output of the patient through the phase difference. As an example, the cardiac output determination module may calculate or otherwise determine cardiac output by simply determining the phase difference, which is then processed to determine cardiac output.

Certain embodiments of the present disclosure provide a tangible and non-transitory computer readable medium that includes one or more sets of instructions configured to direct a computer to emit light from a light emitter of a physiological signal detection unit into vasculature proximate to an anatomical portion of a patient, detect first and second physiological signals with first and second photodetectors, respectively, positioned in relation to first and second locations of the vasculature, receive the first and second physiological signals at a cardiac output determination module, use the cardiac output determination module to determine a phase difference between the first and second physiological signals, and calculate, with the cardiac output determination module, the cardiac output of the patient through the phase difference.

### BRIEF DESCRIPTION OF THE DRAWINGS

FIG. 1 illustrates a block diagram of a system for determining cardiac output, according to an embodiment of the present disclosure.

FIG. 2 illustrates a simplified view of a physiological signal detection unit configured to be secured to a finger of an individual, according to an embodiment of the present disclosure.

FIG. 3 illustrates a simplified view of a physiological signal detection unit configured to be secured to or, placed on, skin of an individual, according to an embodiment of the present disclosure.

FIG. 4 illustrates a simplified view of a physiological signal detection unit configured to be worn around a body part of an individual, according to an embodiment of the present disclosure.

FIG. 5 illustrates a simplified view of a physiological signal detection unit configured to be worn on a head of an individual, according to an embodiment of the present disclosure.

FIG. 6 illustrates a simplified view of a physiological signal detection unit configured to be worn around a body part of an individual, according to an embodiment of the present disclosure.

FIG. 7 illustrates a front view of a physiological signal detection unit secured to a finger, according to an embodiment of the present disclosure.

FIG. 8 illustrates a front view of a physiological signal detection unit secured to a forehead, according to an embodiment of the present disclosure.

FIG. 9 illustrates a front view of a physiological signal detection unit aligned in relation to an artery within a neck, according to an embodiment of the present disclosure.

FIG. 10 illustrates a front view of a physiological signal detection unit aligned in relation to an artery within a forearm, according to an embodiment of the present disclosure.

FIG. 11 illustrates a photoplethysmogram (PPG) signal over time, according to an embodiment of the present disclosure.

FIG. 12 illustrates first and second PPG signals detected by first and first and second photodetectors over time, according to an embodiment of the present disclosure.

FIG. 13 illustrates a flow chart of a method of non-invasively determining cardiac output, according to an embodiment of the present disclosure.

FIG. 14 illustrates an isometric view of a photoplethysmogram system, according to an embodiment of the present disclosure.

FIG. 15 illustrates a simplified block diagram of a PPG system, according to an embodiment of the present disclosure.

### DETAILED DESCRIPTION

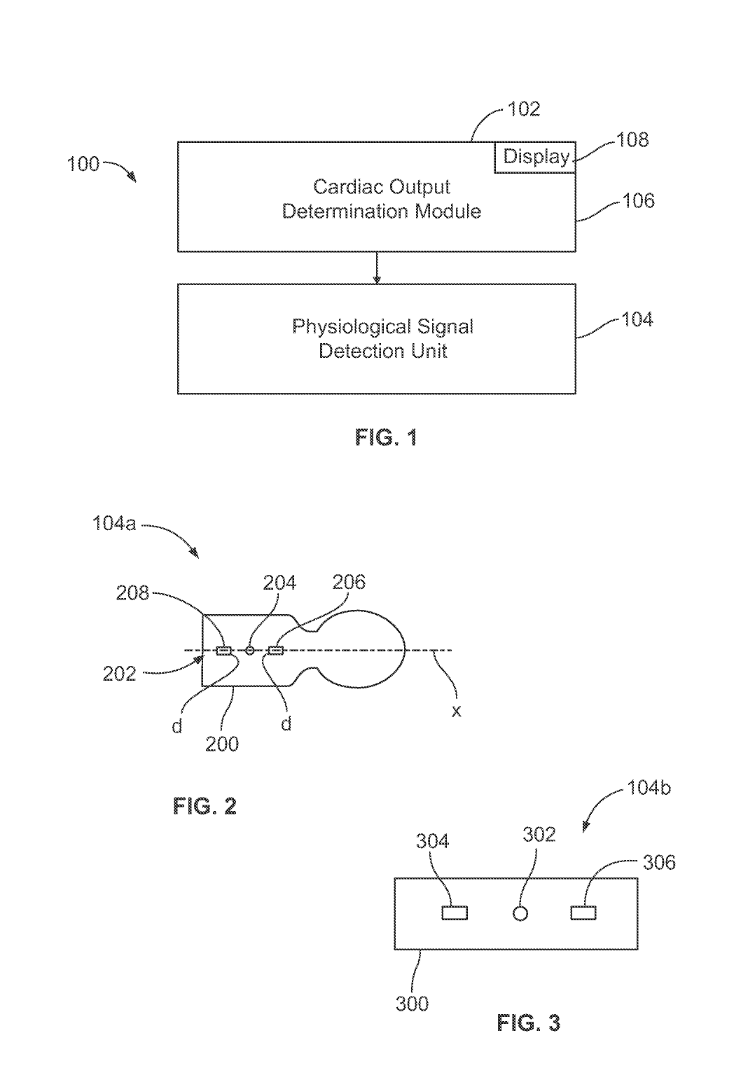

FIG. 1 illustrates a block diagram of a system 100 for determining cardiac output, according to an embodiment of the present disclosure. The system 100 may include a cardiac output determination module 102 operatively connected to a physiological signal detection unit 104, which may include a patient-engaging device (such as a band, headset, housing, or the like having an emitter and multiple photodetectors) configured to secure to or otherwise be positioned on an anatomical structure of a patient. The cardiac output determination module 102 may be operatively connected to the physiological signal detection unit 104 through cables, wireless connections, and/or the like.

The cardiac output determination module 102 may be contained within a workstation 106 that may be or otherwise include one or more computing devices, such as standard computer hardware. The workstation 106 may include one or more modules and control units, such as processing devices that may include one or more microprocessors, microcontrollers, integrated circuits, memory, such as read-only and/or random access memory, and the like. Optionally, the cardiac output determination module 102 may not be contained within a workstation, but, instead, simply include computing circuitry and the like contained within a housing, such as that of the detection unit 104. The cardiac output determination module 102 may be configured to analyze one or more physiological signals or waveforms received from the physiological signal detection unit 104 in order to determine a cardiac output of a patient wearing or otherwise connected to the physiological signal detection unit 104. The signal(s) or waveform(s) may be photoplethysmography (PPG), pulse oximetry, electrocardiograph, or various other signals or waveforms.

While shown as separate and distinct modules, the cardiac output determination module 102 and the physiological signal detection unit 104 may alternatively be integrated into a single housing or module having a processor, controller, integrated circuit or the like. For example, the cardiac output determination module 102 and the physiological signal detection unit 104 may be contained within a single band, strip, bandage, or the like that is configured to be secured to a portion of an individual's body. For example, the determination module 102 and the detection unit 104 may be contained within a head band or strap that is configured to be secured or otherwise placed on a head of the individual.

The workstation 106 may also include a display 108, such as a cathode ray tube display, a flat panel display, such as a liquid crystal display (LCD), light-emitting diode (LED) display, a plasma display, or any other type of monitor. The workstation 106 may be configured to calculate physiological parameters and to show information related to cardiac output on the display 108. For example, the workstation 106 may be configured to display cardiac output, an estimate of a patient's blood oxygen saturation generated by a pulse oximeter (referred to as an SpO₂ measurement), and blood pressure on the display 108.

The cardiac output determination module 102 may include any suitable computer-readable media used for data storage. Computer-readable media are configured to store information that may be interpreted by the cardiac output determination module 102. The information may be data or may take the form of computer-executable instructions, such as software applications, that cause a microprocessor or other such control unit within the cardiac output determination module 102 to perform certain functions and/or computer-implemented methods. The computer-readable media may include computer storage media and communication media. The computer storage media may include volatile and non-volatile media, removable and non-removable media implemented in any method or technology for storage of information such as computer-readable instructions, data structures, program modules or other data. The computer storage media may include, but are not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which may be used to store desired information and that may be accessed by components of the system.

As noted, in operation, the cardiac output determination module 102 receives one or more signals from the physiological signal detection unit 104. The cardiac output determination module 102 analyzes the received signals to detect cardiac output of an individual. The cardiac output determination module 102 is configured to non-invasively determine cardiac output, or blood volumetric flow rate, by analyzing a phase difference between two signals, such as PPG signals, as explained below.

FIG. 2 illustrates a simplified view of a physiological signal detection unit 104a configured to be secured to a finger of an individual, such as a patient within a medical facility, according to an embodiment of the present disclosure. The detection unit 104a includes a housing 200 defining an internal chamber 202 configured to receive a finger of the individual. A light emitter 204 is secured to the housing 200 and is configured to emit light radiation at one or more wavelengths into the finger. A first photodetector 206 is positioned a distance d on one side of the light emitter 204, while a second photodetector 208 is positioned a distance d on an opposite side of the light emitter 204. As such, the first and second photodetectors 206 and 208 may be equidistant from the light emitter 204. The photodetectors 206, 208 and the light emitter 204 may be linearly aligned along a longitudinal axis X of the housing 200. Alternatively, the photodetectors 206, 208 and the light emitter 204 may be oriented at different angles with respect to one another. Also, alternatively, the photodetectors 206 and 208 may not be equidistant from the light emitter 204.

In general, the photodetectors 206, 208 and the light emitter 204 are configured to be aligned with vasculature within the finger. For example, the photodetectors 206, 208 and the light emitter 204 may be positioned over and along the same artery or vein within the finger. As shown, the first photodetector 206 may be positioned closer to a fingertip than the second photodetector 208. Accordingly, the photodetectors 206 and 208 are configured to detect light emitted from the light emitter 204 at different points of the vasculature within the finger.

In operation, the housing 200 is positioned on a finger such that the photodetectors 206, 208 and the light emitter 204 are aligned along common vasculature, such as an artery or vein within the finger. The light emitter 204 emits light radiation, which is detected by each of the photodetectors 206 and 208. Each photodetector 206 and 208 may detect light energy at a particular wavelength. For example, each photodetector 206 and 208 may be configured to detect light at a wavelength corresponding to red light, or infrared light. The photodetectors 206 and 208 may be similarly-configured to detect light at the same wavelength, or the first photodetector 206 may be configured to detect light at a first wavelength, while the second photodetector 206 may be configured to detect light at a second wavelength that differs from the first wavelength.

The photodetectors 206 and 208 detect the light emitted from the light emitter 204 and reflected from blood circulating through the underlying vasculature. The photodetectors 206 and 208 generate and output physiological signals or waveforms, such as PPG signals, that are then sent to the cardiac output determination module 102 (shown in FIG. 1), which analyzes the physiological signals or waveforms and calculates or otherwise determines cardiac output from the received physiological signals or waveforms.

As noted, the detection unit 104a may be separate and distinct from the cardiac output determination module 102. However, the cardiac output determination module 102 may be integrally formed with the detection unit 102. For example, the housing 200 may contain the cardiac output determination module 102.

While the detection unit 104a is shown as being configured to be positioned on a finger of a patient, the detection unit 104a may be sized and shaped differently, and configured to be positioned with respect to other patient anatomy, such as an arm, neck, forehead, or the like.

FIG. 3 illustrates a simplified view of a physiological signal detection unit 104b configured to be secured to or, placed on, skin of a patient, according to an embodiment of the present disclosure. The detection unit 104b may be formed of a flexible strap 300, such as an elastomeric strap, bandage, strip, or the like, that is configured to be positioned on an anatomical structure of a patient, such as a forehead. The flexible strap 300 supports a light emitter 302 and first and second photodetectors 304 and 306, as described above. The flexible strap 300 is configured to be positioned on the patient anatomy and aligned with respect to underlying vasculature, such as a carotid artery, vein in the forehead, femoral artery, or the like.

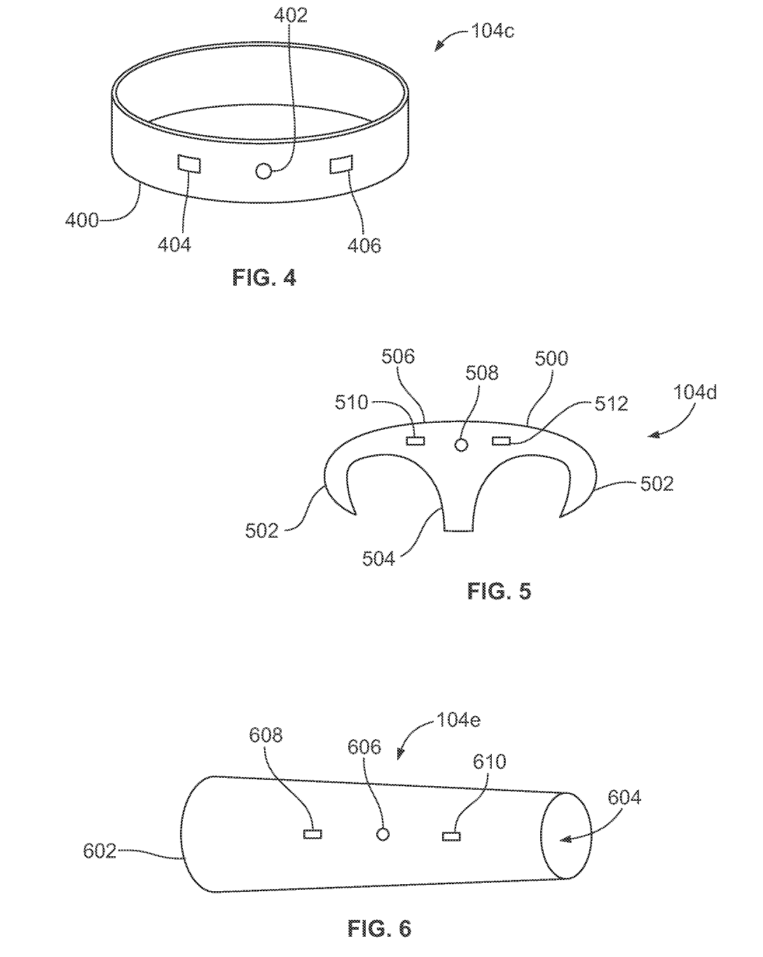

FIG. 4 illustrates a simplified view of a physiological signal detection unit 104c configured to be worn around a body part of a patient, according to an embodiment of the present disclosure. The detection unit 104c may be formed of an annular flexible band 400, such as an elastomeric headband, that is configured to be positioned on patient, anatomy. The flexible band 400 supports a light emitter 402 and first and second photodetectors 404 and 406, as described above. The flexible band 400 is configured to be positioned on patient anatomy and aligned with respect to underlying vasculature, such as a vein or artery in the forehead.

FIG. 5 illustrates a simplified view of a physiological signal detection unit 104d configured to be worn on a head of a patient, according to an embodiment of the present disclosure. The detection unit 104d may be a headset 500 having opposed lateral supports 502 connected to a nose support 504 by an upper band 506. The headset 500 is configured to be positioned on a head of a patient, such that the nose support 504 is positioned over a portion of a nose, and the lateral supports 502 are positioned on sides of the patient's head. In this manner, the headset 500 is configured to be reliably and reputably positioned in a similar orientation on the heads time and time again. The headset 500 is configured to be repeatedly positioned with respect to the head so that a light emitter 508 and photodetectors 510 and 512 are located at the same position with a high degree of accuracy. While shown on the upper band 506, the light emitter 508 and the photodetectors 510 and 512 may be positioned at various other locations of the detection unit 104d. Additional light emitters and photodetectors may also be secured to the detection unit 104d.

FIG. 6 illustrates a simplified view of a physiological signal detection unit 104e configured to be worn around a body part of a patient, according to an embodiment of the present disclosure. The detection unit 104e may be a flexible sleeve or cuff 602 defining an internal passage 604. The sleeve or cuff 602 may be positioned over a forearm, shin, thigh, or the like. Similar to the other detection units, the detection unit 104e includes a light emitter 606 and photodetectors 608 and 610 configured to be aligned with respect to patient vasculature.

Referring to FIGS. 1-6, the physiological signal detection unit 104, examples of which are shown in FIGS. 2-6, may include more light emitters and/or photodetectors than shown. Further, each set may or may not be linearly aligned.

The physiological signal detection unit 104, such as any of those shown in FIGS. 2-6, may include a coupling agent (not shown) that is configured to allow the transmission of both acoustic energy and light therethrough. The coupling agent may be any type of coupling agent that is configured to allow the transmission of both acoustic energy and light therethrough, such as, but not limited to, a gel media, a cream, a fluid, a paste, an ointment, an ultrasound gel, and/or the like. In some embodiments, the physiological signal detection unit 104 may include a sponge (not shown) or other matrix device that is impregnated with the coupling agent for holding the coupling agent. Exemplary coupling agents and housings configured for use as physiological signal detection units are described in U.S. patent application Ser. No. 13/612,160, filed on Sep. 12, 2012, entitled “Photoacoustic Sensor System,” which is hereby incorporated by reference in its entirety.

The physiological signal detection unit 104 may include an adhesive configured to affix the physiological signal detection unit 104 to skin of a patient. The adhesive may thus further secure the physiological signal detection unit 104 in position with respect to patient anatomy. Any type of adhesive may be used. In some embodiments, the adhesive is an adhesive that is specifically designed to adhere to human skin. Moreover, in addition or alternative to the adhesive, the physiological signal detection unit 104 may be configured to be affixed to the patient's skin using any other suitable affixing structure, such as, but not limited to, using suction, an intermediate bracket that is affixed to the patient's skin (using any suitable affixing structure) and is configured to hold the physiological signal detection unit 104, and/or the like. In some alternative embodiments, no affixing structure is used besides the physiological signal detection unit 104 itself. For example, the physiological signal detection unit may include a housing having an ear clip configured to hold the physiological signal detection unit 104 with respect to a temporal artery, as described in U.S. patent application Ser. No. 13/618,227, filed on Sep. 14, 2012, entitled “Sensor System,” which is hereby incorporated by reference in its entirety.

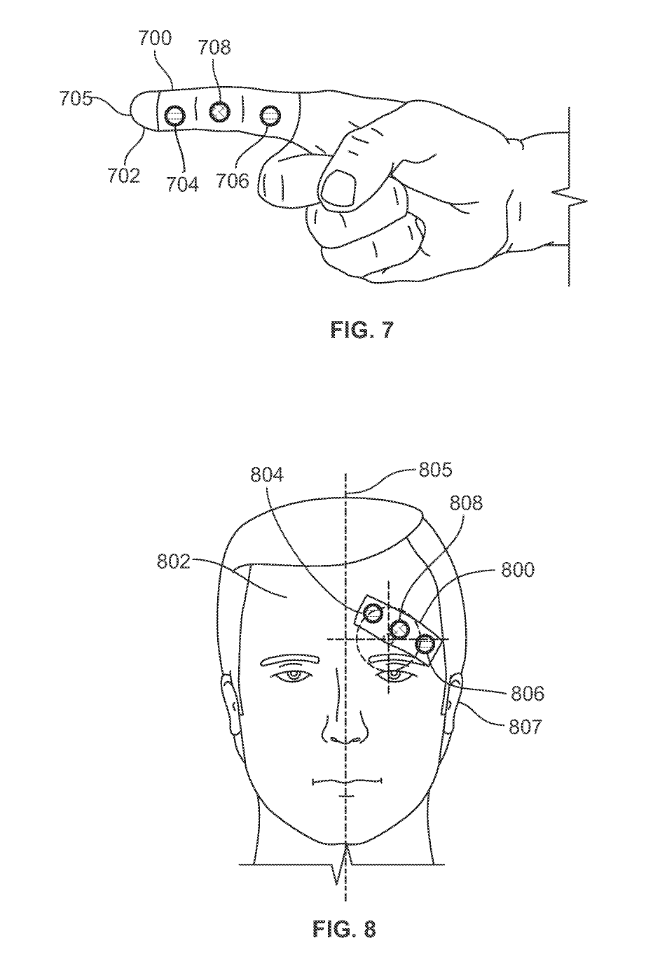

FIG. 7 illustrates a front view of a physiological signal detection unit 700 secured to a finger 702, according to an embodiment of the present disclosure. The physiological signal detection unit 700 may be similar to the detection unit 104a shown in FIG. 2. As shown, a first photodetector 704 is positioned proximate to a fingertip 705, while a second photodetector 706 is distally located from the fingertip 705. A light emitter 708 is positioned between the photodetectors 704 and 706. Each photodetector 704 and 706 may be equidistant from the light emitter 708. The light emitter 708 is configured to emit light into vasculature within the finger. The emitted light is reflected off blood flowing through the vasculature and detected by the photodetectors 704 and 706, which are at different points along the vasculature.

FIG. 8 illustrates a front view of a physiological signal detection unit 800 secured to a forehead 802, according to an embodiment of the present disclosure. The physiological signal detection unit 800 may be similar to the detection unit 104b shown in FIG. 3. As shown, a first photodetector 804 may be positioned proximate to a central axis 805 of the forehead 802, while a second photodetector 806 may be positioned closer to an ear 807. A light emitter 808 is positioned between the photodetectors 804 and 806. Each photodetector 804 and 806 may be equidistant from the light emitter 808. The light emitter 808 is configured to emit light into vasculature within the forehead 802. The emitted light is reflected off blood flowing through the vasculature and detected by the photodetectors 804 and 806, which are at different points along the vasculature. The detection unit 800 may be removed and repositioned with respect to the forehead 802 in various different orientations.

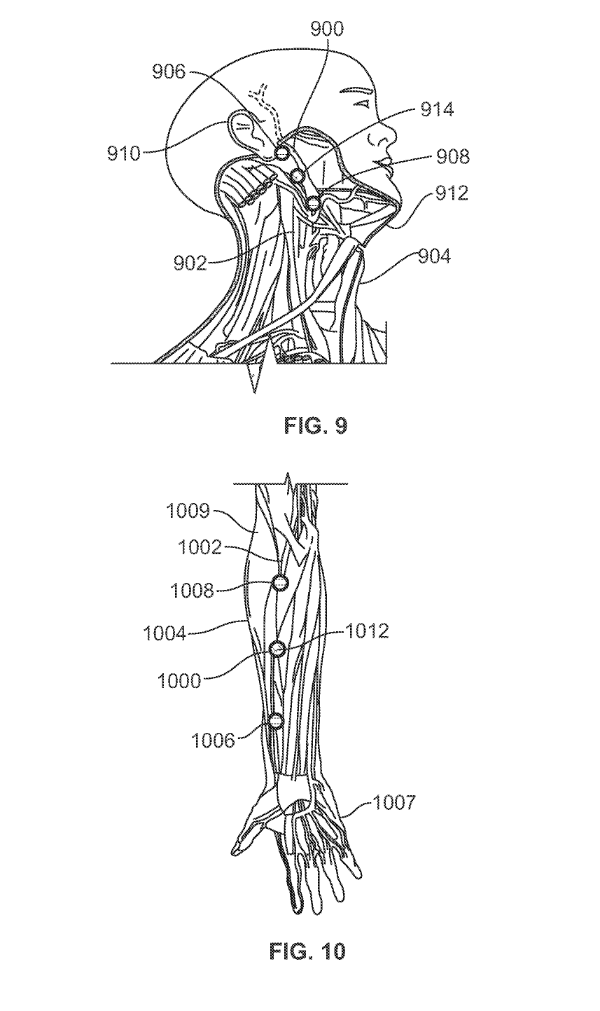

FIG. 9 illustrates a front view of a physiological signal detection unit 900 aligned in relation to an artery 902 within a neck 904, according to an embodiment of the present disclosure. The physiological signal detection unit 900 may be similar to the detection unit 104b shown in FIG. 3. As shown, a first photodetector 906 may be positioned proximate to an ear 910, while a second photodetector 908 may be positioned proximate to a chin 912. A light emitter 914 is positioned between the photodetectors 906 and 908. Each photodetector 906 and 908 may be equidistant from the light emitter 914. The light emitter 914 is configured to emit light into the artery 902, such as a carotid artery. The emitted light is reflected off blood flowing through the artery 902 and detected by the photodetectors 906 and 908, which are at different points along the artery 902. The detection unit 900 may be removed and repositioned with respect to the patient in various different orientations.

FIG. 10 illustrates a front view of a physiological signal detection unit 1000 aligned in relation to an artery 1002 within a forearm 1004, according to an embodiment of the present disclosure. The physiological signal detection unit 1000 may be similar to the detection unit 104e shown in FIG. 6. As shown, a first photodetector 1006 may be positioned proximate to a hand 1007, while a second photodetector 1008 may be positioned proximate to an elbow 1009. A light emitter 1012 is positioned between the photodetectors 1006 and 1008. Each photodetector 1006 and 1008 may be equidistant from the light emitter 1012. The light emitter 1012 is configured to emit light into the artery 1002. The emitted light is reflected off blood flowing through the artery 1002 and detected by the photodetectors 1006 and 1008, which are at different points along the artery 1002. The detection unit 1000 may be removed and repositioned with respect to the patient in various different orientations.

Referring to FIGS. 1-10, a physiological signal detection unit 104 may be positioned at various portions of patient anatomy. In general, the physiological signal detection unit 104 may be positioned over the skin of the patient, such that it may non-invasively detect physiological signals or waveforms from light reflecting from blood flowing through vasculature. That is, the physiological signal detection unit 104 may not be subcutaneously or percutaneously implanted or otherwise surgically inserted into vasculature, tissues, or organs. Instead, the physiological signal detection unit 104 is placed on or over skin of an individual. Alternatively, the physiological signal detection unit 104 may be surgically implanted into patient anatomy. While FIGS. 2-10 show physiological signal detection units configured for use with respect to a finger, forehead, neck, and forearm, it is to be understood that physiological signal detection units may be positioned with respect to various other patient anatomy, as well.

Referring again to FIG. 1, the system 100, including the cardiac output determination module 102 and the physiological signal detection unit 104 (which may be or include any of the detection units shown in FIGS. 2-10), may be configured for PPG detection and analysis. Photoplethysmography (PPG) is a non-invasive, optical measurement that may be used to detect changes in blood volume within tissue, such as skin, of an individual. PPG may be used with pulse oximeters, vascular diagnostics, and digital blood pressure detection systems. A PPG system includes a light source, such as any of the light emitters described above, which is used to illuminate tissue of a patient. The photodetectors are then used to measure small variations in light intensity associated with blood volume changes proximal to the illuminated tissue.

In general, a PPG signal is a physiological signal that includes an AC physiological component related to cardiac synchronous changes in the blood volume with each heartbeat. The AC component is typically superimposed on a DC baseline that may be related to respiration, sympathetic nervous system activity, and thermoregulation.

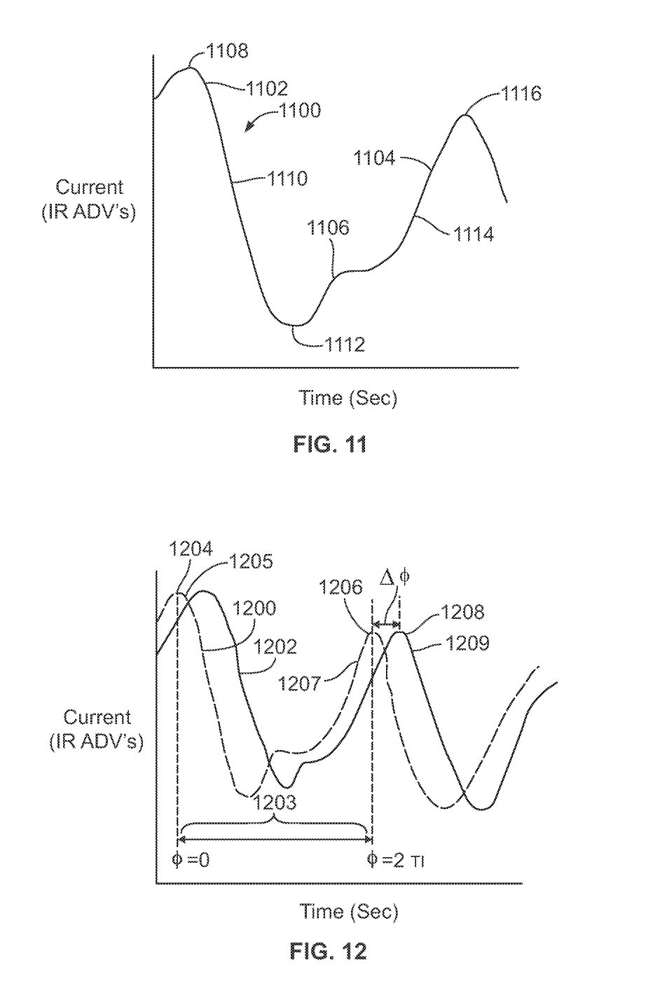

FIG. 11 illustrates a PPG signal 1100 over time, according to an embodiment of the present disclosure. The PPG signal 1100 is an example of a physiological signal. However, embodiments of the present disclosure may be used in relation to various other physiological signals, such as electrocardiogram signals, phonocardiogram signals, ultrasound signals, and the like. Referring to FIGS. 1 and 11, the PPG signal 1100 may be determined, formed, and displayed as a waveform by a display, such as the display 108, which receives signal data from the physiological signal detection unit 104. For example, the cardiac output determination module 102 may receive signals from the physiological signal detection unit 104 positioned on patient anatomy. The cardiac output determination module 102 may process the received signals, and display the resulting PPG signal 1100 on the display 108. Alternatively, the cardiac output determination module 102 may not include the display 108. Instead, the cardiac output determination module 102 may receive and analyze PPG signals from the physiological signal detection unit 104 without displaying the PPG signals.

The PPG signal 1100 may include a plurality of pulses over a predetermined time period. The time period may be a fixed time period, or the time period may be variable. Moreover, the time period may be a rolling time period, such as a 5 second rolling timeframe.

Each pulse may represent a single heartbeat and may include a pulse-transmitted or primary peak 1102 separated from a pulse-reflected or trailing peak 1104 by a dichrotic notch 1106. The primary peak 1102 represents a pressure wave generated from the heart to the point of detection, such as in a finger, forehead, forearm, neck, or the like, where the physiological signal detection unit 104 is positioned. The trailing peak 1104 may represent a pressure wave that is reflected from the location proximate to where the physiological signal detection unit 104 is positioned back toward the heart.

The cardiac output determination module 102 may detect blood pressure based on an analysis of the PPG signal 1100. For example, the cardiac output determination module 102 may map blood pressure to the PPG signal 1100, such that the top 1108 of the primary peak 1102 represents diastolic volume, while descent 1110 of the primary peak 1102 towards the dichrotic notch 1106 represents a systolic rise. A bottom 1112 of the dichrotic notch 1106 may represent systolic peak volume, while an ascent 1114 towards a top 1116 of the trailing peak 1106 may represent a diastolic fall. Accordingly, the PPG signal 1100 may be mapped to blood pressure, and the cardiac output determination module 102 may analyze the PPG signal 1100 to determine the blood pressure.

FIG. 12 illustrates first and second PPG signals 1200 and 1202 detected by first and first and second photodetectors, respectively, over time, according to an embodiment of the present disclosure. The photodetectors may be the first and second photodetectors 704 and 706, for example, as shown in FIG. 7, or any of the photodetectors described above.

A period 1203 of the PPG signal 1200, for example, may be from phase φ=0 to 2π. For example, the period 1203 for the PPG signal 1200 occurs between a top 1204 of primary peak 1205 to a top 1206 of trailing peak 1207. A phase difference Δφ between the PPG signals 1200 and 1202 may be a time difference between the top 1206 of the trailing peak 1207 of the PPG signal 1200, and a top 1208 of a trailing peak 1209 of the PPG signal 1202. However, the phase difference Δφ may be measured between any two corresponding portions of the PPG signals 1200 and 1202, such as between corresponding dichrotic notches, primary peaks, or the like.

Referring again to FIGS. 1 and 12, the cardiac output determination module 102 is configured to determine cardiac output, or blood volumetric flow rate, through a detection of the phase difference Δφ between the PPG signals 1200 and 1202. The physiological signal detection unit 104 detects the physiological signals, such as the PPG signals 1200 and 1202, which the cardiac output determination module 102 analyzes to determine the phase difference Δφ. Based on the phase difference Δφ, the cardiac output determination module 102 is able to automatically calculate or otherwise determine the cardiac output Q, as described below.

Initially, the PPG signals 1200 and 1202 are detected by the physiological signal detection unit 104. The PPG signals 1200 are detected by two photodetectors positioned with respect to two different points along a particular vasculature, such as an artery, vein, or the like, as shown, for example, in FIGS. 7-10. The cardiac output determination module 102 receives the PPG signals 1200 and 1202 from the physiological signal detection unit 104, and analyzes the PPG signals to determine the phase difference Δφ between the two PPG signals 1200 and 1202.

The cardiac output determination module 102 may determine the phase velocity, or pulse wave velocity, as set forth in Equation 1:

$${c}^{′} = \frac{ωΔ z}{Δφ}$$

where c′ is the phase velocity, ω is the heart rate, Δφ is the phase difference, and Δz is the distance between the photodetectors. The heart rate may be determined through an analysis of one or both of the PPG signals 1200 or 1202. Optionally, each detection unit may include a pressure transducer, such as a piezoelectric transducer, configured to detect heart rate. Alternatively, the heart rate may be determined through a separate and distinct heart rate detection module or system. Also, alternatively, if the phase velocity is known, then heart rate may be determined through Equation 1. The cardiac output determination module 102 determines the phase difference Δφ based on a comparison of the PPG signals 1200 and 1202, as described with respect to FIG. 12, for example.

Cardiac output, or blood volumetric flow rate, Q may be calculated in terms of an artery radius R, fluid (blood) density ρ, the phase difference Δφ, which may be measured at two points on vasculature, as described above, Δz, which is the distance between the photodetectors with respect to the vasculature, |P₁|, which is blood pressure, and ω, which is the known or detected heart rate, as shown in Equation 2:

$$Q = \frac{π {R}^{2}}{ωρ} \frac{Δφ}{Δ z} \vert {P}_{1} \vert {M}_{10}^{′} sin ( ω t - {φ}_{1} + {ɛ}_{10} + \frac{π}{2} )$$

where M′₁₀ is described in Equation 3, as follows:

$${M}_{10}^{′} = 1 - \frac{\sqrt{2}}{α} + \frac{1}{{α}^{2}}$$

α is described in Equation 4, as follows:

$$α = {R ( \frac{ωρ}{μ} )}^{\frac{1}{2}}$$

and ε₁₀, is described in Equation 5, as follows:

$${ɛ}_{10} = \frac{\sqrt{2}}{α} + \frac{1}{{α}^{2}} + \frac{19}{24} \frac{1}{\sqrt{2 {α}^{3}}}$$

The blood pressure may be determined through an analysis of one or both of the PPG signals 1200 and 1202, as described above with respect to FIG. 11. Alternatively, the blood pressure may be detected through a separate and distinct blood pressure measuring device on the physiological signal detection unit 104, or on or within a separate component, such as a sphygmomanometer.

The viscosity μ of the blood may, like blood density, be known or estimated and stored in a memory of the cardiac output determination module 102. Thus, α, M′₁₀ and ε₁₀, and therefore Q may be calculated for each harmonic term, and the flow curve Q may be synthesized as the sum of the terms. Accordingly, Equation 2 may be rewritten as Equation 6, as follows:

$$Q = \frac{π {R}^{2}}{ω ρ} \frac{Δφ}{Δ z} \vert {P}_{1} \vert β$$

where β=f(ω,t,φ,R,ρ,μ), all of which are typically known or may be easily estimated, with the possible exception of R. The viscosity μ and the density ρ may be known or estimated. For example, the viscosity and the density may be assumed constant at all different fluid velocities. Therefore, the unknown values from Equation 6 may be the phase difference Δφ, the pressure |P₁|, and the Radius R. The physiological signals, such as the PPG signals 1200 and 1202, and the blood pressure |P₁| may be detected by the physiological signal detection unit 104 and output to the cardiac output determination module 102. The cardiac output determination module 102 may analyze the physiological signals to determine the phase difference Δφ.

Because the vasculature may not be a perfectly round duct, but, instead, may be convoluted and irregular, Equation 3 may be rewritten so that G, or hydraulic diameter replaces R, as follows in Equation 7:

$$Q = \frac{π {G}^{2}}{ωρ} \frac{Δφ}{Δ z} \vert {P}_{1} \vert β$$

G represents a geometric description of irregularly-shaped vasculature, such as arteries, arterioles, capillaries, venules, veins, and the like. G may be known or estimated and stored in the memory of the cardiac output determination module 102.

By determining the phase difference Δφ between the PPG signals 1200 and 1202, the cardiac output determination module 102 may calculate or otherwise determine the cardiac output Q of a patient. The phase difference Δφ may be measured in a single branch of vasculature, such as shown in any of FIGS. 7-10. In general, the phase difference Δφ is determined through detecting the offset between the two PPG signals 1200 and 1202, as shown in FIG. 12.

The difference in current between the two photodetectors may be detected. For example, as shown in FIG. 12, the current may be measured in analog digital units (ADUs). The cardiac output determination module 102 may detect the difference in current between the two photodetectors by measuring the difference between the two PPG signals 1200 and 1202 at an instant in time. The phase difference Δφ may be calculated from heart rate determined from the PPG signals 1200 and 1202 that are detected by the physiological signal detection unit 104, or from another source, such as a pulse oximeter or electrocardiograph monitor ECG operatively connected to the cardiac output determination module 102.

As described above, blood pressure may be determined through an analysis of the PPG signals 1200 and 1202 (such as described with respect to FIG. 11). Optionally, the blood pressure may be determined through a separate and distinct blood pressure measuring system that is operatively connected to the cardiac output determination module 102. For example, an arterial line could be positioned within an artery and configured to detect blood pressure.

Referring again to FIG. 12, consider that in addition to allowing the cardiac output determination module 102 to determine the pulse wave velocity c′, the photodetectors (such as shown and described in FIGS. 2-10) provide two separate and distinct signals that may be used as proxies for blood pressure in the vasculature at the respective locations of the photodetectors. Thus, at any instant in time, there are two values for the current blood pressure P_{c1} and P_{c2}, which represent blood pressure as detected at the first and second photodetectors. Thus, the cardiac output Q₁ in relation to the first photodetector may be expressed as Equation 8:

$${Q}_{1} = \frac{π {G}^{2}}{ωρ} \frac{Δφ}{Δ z} \vert {P}_{c 1} \vert β$$

and the cardiac output Q₂ in relation to the second photodetector may be expressed as Equation 9:

$${Q}_{2} = \frac{π {G}^{2}}{ωρ} \frac{Δφ}{Δ z} \vert {P}_{c 2} \vert β$$

Based on the principle of conservation of mass, the summation of Q₁ over one pulse period is generally equal to the summation of Q₂ over the same pulse period, and may be expressed as Equation 10:

$${\sum }_{t + \frac{2 π}{ω}}^{t} {Q}_{1} = {\sum }_{t + \frac{2 π}{ω}}^{t} {Q}_{2}$$

Thus, the G may be measured, detected, or known and stored in the memory of the cardiac output determination module 102. Alternatively, the G may be estimated based on a known typical size of G and stored in the memory of the cardiac output determination module 102. Accordingly, the cardiac output determination module 102 may calculate or otherwise determine cardiac output Q by detecting the phase difference Δφ between two signals or waveforms, such as two separate and distinct PPG signals 1200 and 1202.

The cardiac output may be quickly and easily determined through a non-invasive physiological signal detection unit 104 secured or positioned on a portion of a patient's body. The physiological signal detection unit 104 may include a light emitter that emits light into the vasculature, and photodetectors that detect light at particular wavelengths that is reflected from blood flowing through the vasculature. Because the photodetectors are spaced apart from one another, each photodetector outputs a signal or waveform, which is received by the cardiac output determination module 102. The cardiac output determination module 102 analyzes the separate and distinct signals, such as PPG signals, received from the separate and distinct photodetectors and determines the phase difference Δφ therebetween. After determining the phase difference Δφ, the cardiac output determination module automatically and non-invasively calculates or otherwise determines the cardiac output Q, as described above. Optionally, if the cardiac output Q is independently verified, the precise G may be determined by using the equations noted above to solve for G. In other words, the precise nature of G may be calculated when Q is known.

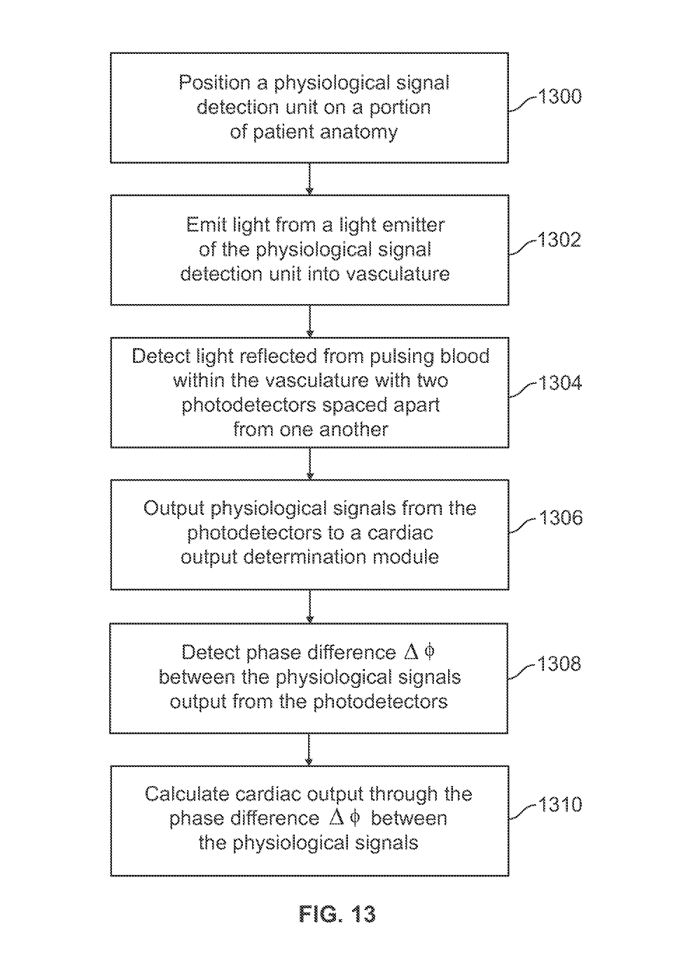

FIG. 13 illustrates a flow chart of a method of non-invasively determining cardiac output, according to an embodiment of the present disclosure. At 1300, a physiological signal detection unit, such as a strip, bandage, strap, headband, headset, sleeve, or the like, is positioned on, or with respect to, a portion of patient anatomy, such as a finger, forehead, arm, leg, thigh, or the like. Once positioned, the physiological signal detection unit is operated to emit light from a light emitter into vasculature of the patient at 1302. Spaced-apart photodetectors of the cardiac output determination unit detect light reflected from pulsing blood within the vasculature at 1304. Then, at 1306, the photodetectors output signals, such as PPG signals, to a cardiac output determination module that is in communication with the physiological signal detection unit. As noted above, the cardiac output determination module and the physiological signal detection unit may be separate and distinct from one another, or may be contained within a common housing or structure.

After receiving the signals from the photodetectors, the cardiac output determination module detects the phase difference Δφ between the signals at 1308. Then, at 1310, the cardiac output determination module calculates or otherwise determines the cardiac output through the phase difference Δφ. For example, by determining the phase difference Δφ, the cardiac output determination module may automatically calculate the cardiac output Q.

Thus, embodiments of the present disclosure provide a system and method of quickly, easily, and non-invasively determining cardiac output.

Embodiments of the present disclosure may be used with respect to a PPG system. For example, the cardiac output determination module 102 (shown in FIG. 1) may be part of a PPG system. Further, the physiological signal detection unit 104 may be or include a PPG sensor.

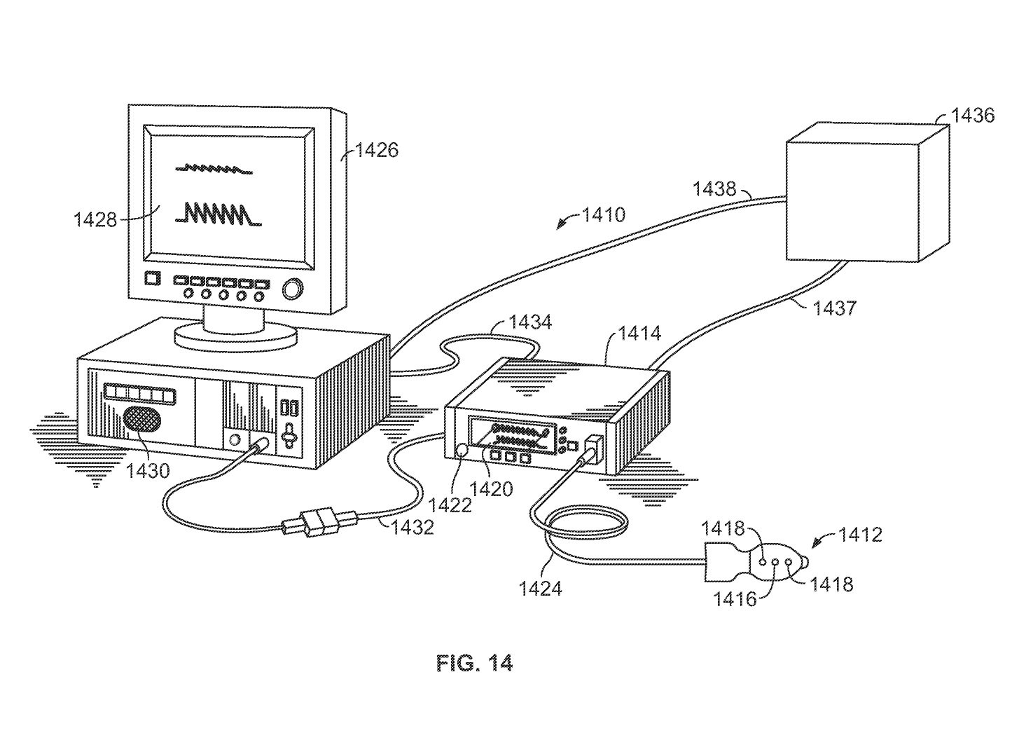

FIG. 14 illustrates an isometric view of a PPG system 1410, according to an embodiment of the present disclosure. While the system 1410 is shown and described as a PPG system 1410, the system may be various other types of physiological detection systems, such as an electrocardiogram system, a phonocardiogram system, and the like. The PPG system 1410 may be a pulse oximetry system, for example. The system 1410 may include a PPG sensor 1412 and a PPG monitor 1414. The PPG sensor 1412 may include an emitter 1416 configured to emit light into tissue of a patient. For example, the emitter 1416 may be configured to emit light at two or more wavelengths into the tissue of the patient. The PPG sensor 1412 may also include spaced-apart photodetectors 1418 that are configured to detect the emitted light from the emitter 1416 that emanates from the tissue after passing through the tissue. The photodetectors 1418 may be equidistant, but on opposite sides, from the emitter 1416.

The system 1410 may include a plurality of sensors forming a sensor array in place of the PPG sensor 1412. Each of the sensors of the sensor array may be a complementary metal oxide semiconductor (CMOS) sensor, for example. Alternatively, each sensor of the array may be a charged coupled device (CCD) sensor. In another embodiment, the sensor array may include a combination of CMOS and CCD sensors. The CCD sensor may include a photoactive region and a transmission region configured to receive and transmit, while the CMOS sensor may include an integrated circuit having an array of pixel sensors. Each pixel may include a photodetector and an active amplifier.

The emitter 1416 and the photodetectors 1418 may be configured to be located on opposite sides of a digit, such as a finger or toe, in which case the light that emanates from the tissue passes completely through the digit. The emitter 1416 and the photodetectors 1418 may be arranged so that light from the emitter 1416 penetrates the tissue and is reflected by the tissue into the detector 1418, such as a sensor designed to obtain pulse oximetry data.

The sensor 1412 or sensor array may be operatively connected to and draw power from the monitor 1414, for example. Optionally, the sensor 1412 may be wirelessly connected to the monitor 1414 and include a battery or similar power supply (not shown). The monitor 1414 may be configured to calculate physiological parameters based at least in part on data received from the sensor 1412 relating to light emission and detection. Alternatively, the calculations may be performed by and within the sensor 1412 and the result of the oximetry reading may be passed to the monitor 1414. Additionally, the monitor 1414 may include a display 1420 configured to display the physiological parameters or other information about the system 1410. The monitor 1414 may also include a speaker 1422 configured to provide an audible sound that may be used in various other embodiments, such as for example, sounding an audible alarm in the event that physiological parameters are outside a predefined normal range.

The sensor 1412, or the sensor array, may be communicatively coupled to the monitor 1414 via a cable 1424. Alternatively, a wireless transmission device (not shown) or the like may be used instead of, or in addition to, the cable 1424.

The system 1410 may also include a multi-parameter workstation 1426 operatively connected to the monitor 1414. The workstation 1426 may be or include a computing sub-system 1430, such as standard computer hardware. The computing sub-system 1430 may include one or more modules and control units, such as processing devices that may include one or more microprocessors, microcontrollers, integrated circuits, memory, such as read-only and/or random access memory, and the like. The workstation 1426 may include a display 1428, such as a cathode ray tube display, a flat panel display, such as a liquid crystal display (LCD), light-emitting diode (LED) display, a plasma display, or any other type of monitor. The computing sub-system 1430 of the workstation 1426 may be configured to calculate physiological parameters and to show information from the monitor 1414 and from other medical monitoring devices or systems (not shown) on the display 1428. For example, the workstation 1426 may be configured to display an estimate of a patient's blood oxygen saturation generated by the monitor 1414 (referred to as an SpO₂ measurement), pulse rate information from the monitor 1414, and blood pressure from a blood pressure monitor (not shown) on the display 1428.

The monitor 1414 may be communicatively coupled to the workstation 1426 via a cable 1432 and/or 1434 that is coupled to a sensor input port or a digital communications port, respectively and/or may communicate wirelessly with the workstation 1426. Additionally, the monitor 1414 and/or workstation 1426 may be coupled to a network to enable the sharing of information with servers or other workstations. The monitor 1414 may be powered by a battery or by a conventional power source such as a wall outlet.

The system 1410 may also include a fluid delivery device 1436 that is configured to deliver fluid to a patient. The fluid delivery device 1436 may be an intravenous line, an infusion pump, any other suitable fluid delivery device, or any combination thereof that is configured to deliver fluid to a patient. The fluid delivered to a patient may be saline, plasma, blood, water, any other fluid suitable for delivery to a patient, or any combination thereof. The fluid delivery device 1436 may be configured to adjust the quantity or concentration of fluid delivered to a patient.

The fluid delivery device 1436 may be communicatively coupled to the monitor 1414 via a cable 1437 that is coupled to a digital communications port or may communicate wirelessly with the workstation 1426. Alternatively, or additionally, the fluid delivery device 1436 may be communicatively coupled to the workstation 1426 via a cable 1438 that is coupled to a digital communications port or may communicate wirelessly with the workstation 1426.

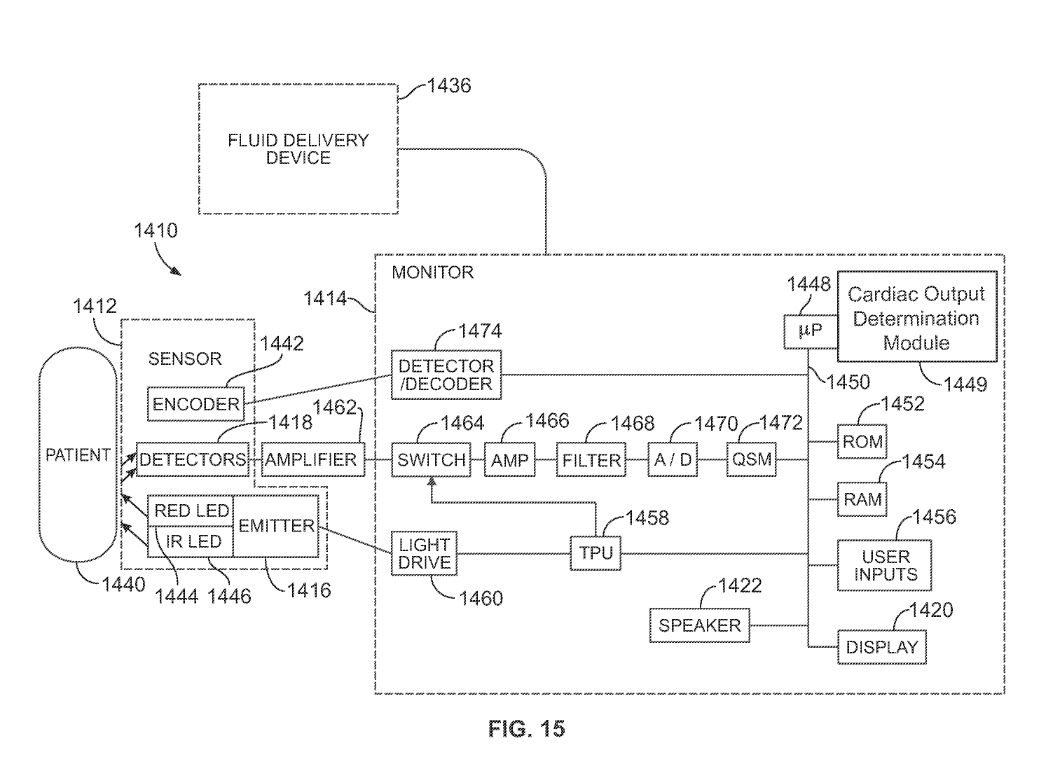

FIG. 15 illustrates a simplified block diagram of the PPG system 1410, according to an embodiment of the present disclosure. When the PPG system 1410 is a pulse oximetry system, the emitter 1416 may be configured to emit at least two wavelengths of light (for example, red and infrared) into tissue 1440 of a patient. Accordingly, the emitter 1416 may include a red light-emitting light source such as a red light-emitting diode (LED) 1444 and an infrared light-emitting light source such as an infrared LED 1446 for emitting light into the tissue 1440 at the wavelengths used to calculate the patient's physiological parameters. For example, the red wavelength may be between about 600 nm and about 700 nm, and the infrared wavelength may be between about 800 nm and about 1000 nm. In embodiments where a sensor array is used in place of single sensor, each sensor may be configured to emit a single wavelength. For example, a first sensor may emit a red light while a second sensor may emit an infrared light.

As discussed above, the PPG system 1410 is described in terms of a pulse oximetry system. However, the PPG system 1410 may be various other types of systems. For example, the PPG system 1410 may be configured to emit more or less than two wavelengths of light into the tissue 1440 of the patient. Further, the PPG system 1410 may be configured to emit wavelengths of light other than red and infrared into the tissue 1440. As used herein, the term “light” may refer to energy produced by radiative sources and may include one or more of ultrasound, radio, microwave, millimeter wave, infrared, visible, ultraviolet, gamma ray or X-ray electromagnetic radiation. The light may also include any wavelength within the radio, microwave, infrared, visible, ultraviolet, or X-ray spectra, and that any suitable wavelength of electromagnetic radiation may be used with the system 1410. The photodetectors 1418 may be configured to be specifically sensitive to the chosen targeted energy spectrum of the emitter 1416.

The photodetectors 1418 may be configured to detect the intensity of light at the red and infrared wavelengths. Alternatively, each sensor in the array may be configured to detect an intensity of a single wavelength. In operation, light may enter the photodetectors 1418 after passing through the tissue 1440. The photodetectors 1418 may convert the intensity of the received light into electrical signals. The light intensity may be directly related to the absorbance and/or reflectance of light in the tissue 1440. For example, when more light at a certain wavelength is absorbed or reflected, less light of that wavelength is received from the tissue by the photodetectors 1418. After converting the received light to an electrical signal, the photodetectors 1418 may send the signal to the monitor 1414, which calculates physiological parameters based on the absorption of the red and infrared wavelengths in the tissue 1440.

In an embodiment, an encoder 1442 may store information about the sensor 1412, such as sensor type (for example, whether the sensor is intended for placement on a forehead or digit) and the wavelengths of light emitted by the emitter 1416. The stored information may be used by the monitor 1414 to select appropriate algorithms, lookup tables and/or calibration coefficients stored in the monitor 1414 for calculating physiological parameters of a patient. The encoder 1442 may store or otherwise contain information specific to a patient, such as, for example, the patient's age, weight, diagnosis, vasculature value G, and/or the like. The information may allow the monitor 1414 to determine, for example, patient-specific threshold ranges related to the patient's physiological parameter measurements, and to enable or disable additional physiological parameter algorithms. The encoder 1442 may, for instance, be a coded resistor that stores values corresponding to the type of sensor 1412 or the types of each sensor in the sensor array, the wavelengths of light emitted by emitter 1416 on each sensor of the sensor array, and/or the patient's characteristics. Optionally, the encoder 1442 may include a memory in which one or more of the following may be stored for communication to the monitor 1414: the type of the sensor 1412, the wavelengths of light emitted by emitter 1416, the particular wavelength each sensor in the sensor array is monitoring, a signal threshold for each sensor in the sensor array, any other suitable information, or any combination thereof.

Signals from the photodetectors 1418 and the encoder 1442 may be transmitted to the monitor 1414. The monitor 1414 may include a general-purpose control unit, such as a microprocessor 1448 connected to an internal bus 1450. The microprocessor 1448 may be configured to execute software, which may include an operating system and one or more applications, as part of performing the functions described herein. A read-only memory (ROM) 1452, a random access memory (RAM) 1454, user inputs 1456, the display 1420, and the speaker 1422 may also be operatively connected to the bus 1450. The control unit and/or the microprocessor 1448 may include a cardiac output determination module 1449 that is configured to determine a cardiac output of a patient, such as through a detected phase difference Δφ between two signals, such as two PPG signals detected by the photodetectors 1418, as described above.

The RAM 1454 and the ROM 1452 are illustrated by way of example, and not limitation. Any suitable computer-readable media may be used in the system for data storage. Computer-readable media are configured to store information that may be interpreted by the microprocessor 1448. The information may be data or may take the form of computer-executable instructions, such as software applications, that cause the microprocessor to perform certain functions and/or computer-implemented methods. The computer-readable media may include computer storage media and communication media. The computer storage media may include volatile and non-volatile media, removable and non-removable media implemented in any method or technology for storage of information such as computer-readable instructions, data structures, program modules or other data. The computer storage media may include, but are not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which may be used to store desired information and that may be accessed by components of the system.

The monitor 1414 may also include a time processing unit (TPU) 1458 configured to provide timing control signals to a light drive circuitry 1460, which may control when the emitter 1416 is illuminated and multiplexed timing for the red LED 1444 and the infrared LED 1446. The TPU 1458 may also control the gating-in of signals from the photodetectors 1418 through an amplifier 1462 and a switching circuit 1464. The signals are sampled at the proper time, depending upon which light source is illuminated. The received signals from the photodetectors 1418 may be passed through an amplifier 1466, a low pass filter 1468, and an analog-to-digital converter 1470. The digital data may then be stored in a queued serial module (QSM) 1472 (or buffer) for later downloading to RAM 1454 as QSM 1472 fills up. In an embodiment, there may be multiple separate parallel paths having amplifier 1466, filter 1468, and A/D converter 1470 for multiple light wavelengths or spectra received.

The microprocessor 1448 may be configured to determine the patient's physiological parameters, such as SpO₂ and pulse rate using various algorithms and/or look-up tables based on the value(s) of the received signals and/or data corresponding to the light received by the photodetectors 1418. Similarly, the cardiac output determination module 1449 may be configured to determine the cardiac output of a patient using various algorithms and/or look-up tables (for example, stored values for G) based on the value(s) of the received signals and/or data received from the photodetectors 1418. The signals corresponding to information about a patient, and regarding the intensity of light emanating from the tissue 1440 over time, may be transmitted from the encoder 1442 to a decoder 1474. The transmitted signals may include, for example, encoded information relating to patient characteristics. The decoder 1474 may translate the signals to enable the microprocessor 1448 to determine the thresholds based on algorithms or look-up tables stored in the ROM 1452. The user inputs 1456 may be used to enter information about the patient, such as age, weight, height, diagnosis, medications, treatments, and so forth. The display 1420 may show a list of values that may generally apply to the patient, such as, for example, age ranges or medication families, which the user may select using the user inputs 1456.

The fluid delivery device 1436 may be communicatively coupled to the monitor 1414. The microprocessor 1448 may determine the patient's physiological parameters, such as a change or level of fluid responsiveness, and display the parameters on the display 1420. In an embodiment, the parameters determined by the microprocessor 1448 or otherwise by the monitor 1414 may be used to adjust the fluid delivered to the patient via fluid delivery device 1436.

As noted, the PPG system 1410 may be a pulse oximetry system. A pulse oximeter is a medical device that may determine oxygen saturation of blood. The pulse oximeter may indirectly measure the oxygen saturation of a patient's blood (as opposed to measuring oxygen saturation directly by analyzing a blood sample taken from the patient) and changes in blood volume in the skin. Ancillary to the blood oxygen saturation measurement, pulse oximeters may also be used to measure the pulse rate of a patient. Pulse oximeters measure and display various blood flow characteristics including, but not limited to, the oxygen saturation of hemoglobin in arterial blood.

A pulse oximeter may include a light sensor, similar to the sensor 1412, that is placed at a site on a patient, typically a fingertip, toe, forehead or earlobe, or in the case of a neonate, across a foot. The pulse oximeter may pass light using a light source through blood perfused tissue and photoelectrically sense the absorption of light in the tissue. For example, the pulse oximeter may measure the intensity of light that is received at the light sensor as a function of time. A signal representing light intensity versus time or a mathematical manipulation of this signal (for example, a scaled version thereof, a log taken thereof, a scaled version of a log taken thereof, and/or the like) may be referred to as the PPG signal. In addition, the term “PPG signal,” as used herein, may also refer to an absorption signal (for example, representing the amount of light absorbed by the tissue) or any suitable mathematical manipulation thereof. The light intensity or the amount of light absorbed may then be used to calculate the amount of the blood constituent (for example, oxyhemoglobin) being measured as well as the pulse rate and when each individual pulse occurs.

The light passed through the tissue is selected to be of one or more wavelengths that are absorbed by the blood in an amount representative of the amount of the blood constituent present in the blood. The amount of light passed through the tissue varies in accordance with the changing amount of blood constituent in the tissue and the related light absorption. Red and infrared wavelengths may be used because it has been observed that highly oxygenated blood will absorb relatively less red light and more infrared light than blood with lower oxygen saturation. By comparing the intensities of two wavelengths at different points in the pulse cycle, it is possible to estimate the blood oxygen saturation of hemoglobin in arterial blood.

The PPG system 1410 and pulse oximetry may be further described in United States Patent Application Publication No. 2012/0053433, entitled “System and Method to Determine SpO₂ Variability and Additional Physiological Parameters to Detect Patient Status,” United States Patent Application Publication No. 2010/0324827, entitled “Fluid Responsiveness Measure,” and United States Patent Application Publication No. 2009/0326353, entitled “Processing and Detecting Baseline Changes in Signals,” all of which are hereby incorporated by reference in their entireties.

It will be understood that the present disclosure is applicable to any suitable physiological signals and that PPG are used for illustrative purposes. Those skilled in the art will recognize that the present disclosure has wide applicability to other signals including, but not limited to other physiological signals (for example, electrocardiogram, electroencephalogram, electrogastrogram, electromyogram, heart rate signals, pathological sounds, ultrasound, or any other suitable biosignal) and/or any other suitable signal, and/or any combination thereof.

Various embodiments described herein provide a tangible and non-transitory (for example, not an electric signal) machine-readable medium or media having instructions recorded thereon for a processor or computer to operate a system to perform one or more embodiments of methods described herein. The medium or media may be any type of CD-ROM, DVD, floppy disk, hard disk, optical disk, flash RAM drive, or other type of computer-readable medium or a combination thereof.

The various embodiments and/or components, for example, the control units, modules, or components and controllers therein, also may be implemented as part of one or more computers or processors. The computer or processor may include a computing device, an input device, a display unit and an interface, for example, for accessing the Internet. The computer or processor may include a microprocessor. The microprocessor may be connected to a communication bus. The computer or processor may also include a memory. The memory may include Random Access Memory (RAM) and Read Only Memory (ROM). The computer or processor may also include a storage device, which may be a hard disk drive or a removable storage drive such as a floppy disk drive, optical disk drive, and the like. The storage device may also be other similar means for loading computer programs or other instructions into the computer or processor.

As used herein, the term “computer” or “module” may include any processor-based or microprocessor-based system including systems using microcontrollers, reduced instruction set computers (RISC), application specific integrated circuits (ASICs), logic circuits, and any other circuit or processor capable of executing the functions described herein. The above examples are exemplary only, and are thus not intended to limit in any way the definition and/or meaning of the term “computer”.

The computer or processor executes a set of instructions that are stored in one or more storage elements, in order to process input data. The storage elements may also store data or other information as desired or needed. The storage element may be in the form of an information source or a physical memory element within a processing machine.

The set of instructions may include various commands that instruct the computer or processor as a processing machine to perform specific operations such as the methods and processes of the various embodiments of the subject matter described herein. The set of instructions may be in the form of a software program. The software may be in various forms such as system software or application software. Further, the software may be in the form of a collection of separate programs or modules, a program module within a larger program or a portion of a program module. The software also may include modular programming in the form of object-oriented programming. The processing of input data by the processing machine may be in response to user commands, or in response to results of previous processing, or in response to a request made by another processing machine.

As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. The above memory types are exemplary only, and are thus not limiting as to the types of memory usable for storage of a computer program.

While various spatial and directional terms, such as top, bottom, lower, mid, lateral, horizontal, vertical, front, and the like may be used to describe embodiments, it is understood that such terms are merely used with respect to the orientations shown in the drawings. The orientations may be inverted, rotated, or otherwise changed, such that an upper portion is a lower portion, and vice versa, horizontal becomes vertical, and the like.

It is to be understood that the above description is intended to be illustrative, and not restrictive. For example, the above-described embodiments (and/or aspects thereof) may be used in combination with each other. In addition, many modifications may be made to adapt a particular situation or material to the teachings without departing from its scope. While the dimensions, types of materials, and the like described herein are intended to define the parameters of the disclosure, they are by no means limiting and are exemplary embodiments. Many other embodiments will be apparent to those of skill in the art upon reviewing the above description. The scope of the disclosure should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means—plus-function format and are not intended to be interpreted based on 35 U.S.C. §112, sixth paragraph, unless and until such claim limitations expressly use the phrase “means for” followed by a statement of function void of further structure.

What is claimed is:

1. A system for non-invasively determining cardiac output of a patient, the system comprising:
a physiological signal detection unit configured to detect first and second physiological signals with respect to first and second locations of vasculature of the patient; and
a cardiac output determination module that is configured to receive the first and second physiological signals and calculate the cardiac output of the patient based, at least in part, on a phase difference between the first and second physiological signals.

2. The system of claim 1, wherein the physiological signal detection unit comprises a light emitter and first and second photodetectors, and wherein the first and second photodetectors are configured to detect the first and second physiological signals, respectively.

3. The system of claim 1, wherein the light emitter and the first and second photodetectors are configured to align with the vasculature of the patient.

4. The system of claim 1, wherein the first and second photodetectors are equidistant from the light emitter.

5. The system of claim 1, wherein each of the first and second physiological signals comprises a photoplethysmography (PPG) signal.

6. The system of claim 1, wherein the physiological signal detection unit comprises a pulse oximetry sensor.

7. The system of claim 1, wherein the physiological signal detection unit comprises a housing defining an internal chamber configured to receive a portion of a finger.

8. The system of claim 1, wherein the physiological signal detection unit comprises a strap configured to be positioned on an anatomical portion of the patient.

9. The system of claim 1, wherein the physiological signal detection unit comprises one or more of a headband or a headset.

10. The system of claim 1, wherein the physiological signal detection unit comprises a sleeve configured to be positioned around a portion of an arm or a leg.

11. A method of non-invasively determining cardiac output of a patient, the method comprising:
positioning a physiological signal detection unit with respect to an anatomical portion of the patient;
emitting light from a light emitter of the physiological signal detection unit into vasculature proximate to the anatomical portion;
detecting first and second physiological signals with first and second photodetectors, respectively, positioned in relation to first and second locations of the vasculature;
receiving the first and second physiological signals at a cardiac output determination module;
using the cardiac output determination module to determine a phase difference between the first and second physiological signals; and
calculating, with the cardiac output determination module, the cardiac output of the patient based, at least in part, on the phase difference between the first and second physiological signals.

12. The method of claim 11, wherein the positioning operation comprises aligning the light emitter and the first and second photodetectors are with the vasculature.

13. The method of claim 11, wherein the positioning comprises spacing the first and second photodetectors an equal distance away from the light emitter.

14. The method of claim 11, wherein each of the first and second physiological signals comprises a photoplethysmography (PPG) signal.

15. The method of claim 11, wherein the anatomical portion of the patient comprises one or more of a finger, forehead, neck, arm, or leg.

16. A tangible and non-transitory computer readable medium that includes one or more sets of instructions configured to direct a computer to:
emit light from a light emitter of a physiological signal detection unit into vasculature proximate to an anatomical portion of a patient;
detect first and second physiological signals with first and second photodetectors, respectively, positioned in relation to first and second locations of the vasculature;
receive the first and second physiological signals at a cardiac output determination module;
use the cardiac output determination module to determine a phase difference between the first and second physiological signals; and
calculate, with the cardiac output determination module, the cardiac output of the patient based, at least in part, on the phase difference between the first and second physiological signals.

17. The tangible and non-transitory computer readable medium of claim 16, wherein the light emitter and the first and second photodetectors are aligned with the vasculature.

18. The tangible and non-transitory computer readable medium of claim 16, wherein the first and second photodetectors are positioned an equal distance away from the light emitter.

19. The tangible and non-transitory computer readable medium of claim 16, wherein each of the first and second physiological signals comprises a photoplethysmography (PPG) signal.

20. The tangible and non-transitory computer readable medium of claim 16, wherein the anatomical portion of the patient comprises one or more of a finger, forehead, neck, arm, or leg.
